(19) United States
(12) Patent Application Publication
(10) Pub. No.: US 2003/0004455 A1
(43) Pub. Date: Jan. 2, 2003

(54) Bi-manual phaco needle

(76) Inventors: Kenneth E. Kadziauskas, Coto de Caza, CA (US); Paul W. Rockley, Laguna Niguel, CA (US); Mark E. Steen, Chino Hills, CA (US)

Correspondence Address: Walter A. Hackler, Ph.D., Attorney of Record, 2372 S.E. Bristol, Suite B., Newport Beach, CA, 92660-0755, US

(21) Appl. No.: 09/894,503

(22) Filed: Jun. 28, 2001

(51) Int. Cl. A61M 1/00

(52) U.S. Cl. 604/27

(57) ABSTRACT

Apparatus for the phacoemulsification of lens tissue includes a first needle for introducing irrigation fluid into a lens capsule and a second ultrasonically vibrated needle for inserting into the lens needle and emulsifying the lens tissue therein. The second needle includes a lumen therethrough for aspiration of emulsified lens tissue and irrigation fluid from the lens capsule through a primary aspiration port defined by an end of the lumen. At least one secondary aspiration port is disposed in the second needle is a spaced apart relationship with the primary aspiration port for introducing emulsified lens and irrigation fluid into the lumen.

The present invention generally relates to surgical needles and more particularly relates to phacoemulsification needles which provide improved irrigation and reduced risk of corneal or scleral tissue damage during cataract removal.

Cataracts cause the lens of an eye to become clouded and a common practice to alleviate this condition is by surgically removing the cataractic lens and replacing it with an artificial interocular lens.

Early lens removal was effected through manual extraction which required a wound of about 12 mm in length. This large opening can result in corneal or scleral tissue damage.

Phacoemulsification enables the removal of a cataractic lens through a much smaller incision, for example between about 2.5 to about 4 mm. In this procedure, a needle is inserted through the incision into a lens capsule and the needle is ultrasonically vibrated to mechanically emulsify the lens. Once fragmented, or emulsified, the lens material is aspirated through a lumen through the phacoemulsification needle.

While emulsifying the lens and aspirating lens fragments, a simultaneous flow of irrigation fluid into the lens capsule is provided around the needle through an annulus established by a sleeve concentrically disposed over the needle. This flow of liquid into the eye is necessary to prevent collapse of the interior chamber of the eye during aspiration. In addition, the irrigation fluid cools the needle in order to prevent any thermal damage of the corneal or scleral tissue. While the sleeve surrounding a phacoemulsification needle provides the important function of establishing an annulus for introducing irrigation fluid into the lens capsule and also enlarges the overall diameter of the sleeve needle for which an incision must be made.

In addition, when irrigation fluid is introduced proximate the emulsifying needle tip, the immediate area in front of the needle is roiled. This occurs because of the counter-current flow of fluid being aspirated by the needle itself and the irrigation fluid being introduced over the surface of the needle. Needle vibration causes a cloud of debris which is roiled by the incoming infusion fluid which lessons the physicians visual acuity of the end of the needle which can slow the procedure.

The present invention overcomes the drawbacks of a sleeved phacoemulsification needle.

### SUMMARY OF THE INVENTION

Apparatus for the phacoemulsification of lens tissue in accordance with the present invention generally includes a first needle for introducing an irrigation fluid into a lens capsule and a second ultrasonically vibrated needle for inserting into the lens capsule for emulsifying the lens tissue therein. The second needle includes a lumen therethrough for aspiration of a emulsified lens tissue and irrigation fluid from the lens capsule through a primary aspiration port defined by an end of the lumen.

Also provided is at least one secondary aspiration port disposed in the second needle in a spaced apart relationship with the primary aspiration port for introducing emulsified lens and irrigation fluid into the lumen. Because the apparatus in accordance with the present invention does not include a sleeve surrounding the ultrasonically vibrated needle, a smaller incision or wound is required in the cornea or sclera.

In addition, because irrigation fluid is not simultaneous introduced proximate the second emulsifying needle, no disturbance or churning of fluids occurs which may provide for a “milky cloud” at the end of the needle which may tend to lesson visual acuity which may interfere with the accuracy of the phacoemulsification by a physician.

More particularly, the cross-sectional area of the secondary aspiration port may be up to 40% of a cross-sectional area of the primary aspiration port. This insures that a dominant portion of the aspirated fluid occurs through the primary aspiration port. If the primary aspiration port becomes occluded, the secondary aspiration port maintains fluid flow through the needle to insure cooling thereof to prevent any overheating of cornea or scleral tissue.

More particularly, the secondary aspiration port may be disposed within 7 mm of the primary aspiration port to insure that both ports are present in the lens capsule during phacoemulsification procedure.

Alternatively, the apparatus in accordance with the present invention may include multiple secondary aspiration ports in the second needle and a total cross-sectional of the multiple secondary aspiration ports may be up to 40% of the cross-sectional area of the primary aspiration port. All of the multiple secondary aspiration ports may be disposed within 9 mm of the primary aspiration port.

In one embodiment of the present invention, the multiple secondary aspiration ports may be disposed in a spaced apart radial relationship with one another circumferally around the second needle.

### BRIEF DESCRIPTION OF THE DRAWINGS

The advantages and features of the present invention will be better understood by the following description when considered in conjunction with the accompanying drawings in which:

FIG. 1 is a diagram of apparatus in accordance with the present invention generally showing a first needle for introducing an irrigation fluid into a lens capsule through a handpiece from an operating console along with a secondary ultrasonically vibrated needle for inserting into the lens capsule and operated by a phacoemulsification handpiece controlled by the console through an aspiration line and an ultrasonic power line;

FIG. 2 is a view of the ultrasonically vibrated needle shown in FIG. 1 showing a primary aspiration port along with two secondary aspiration ports formed in the needle in a spaced apart distance from the primary aspiration port;

FIG. 3 is a view of the needle tip shown in FIG. 2 illustrating non turbulent aspiration of lens tissue and irrigation fluid; and

FIG. 4 is a similar view of a prior art sleeved needle top illustrating turbulence or roiling of fluid in front of the needle tip caused by countercurrent fluid flow with results in a cloud of emulsified lens tissue.

### DETAILED DESCRIPTION

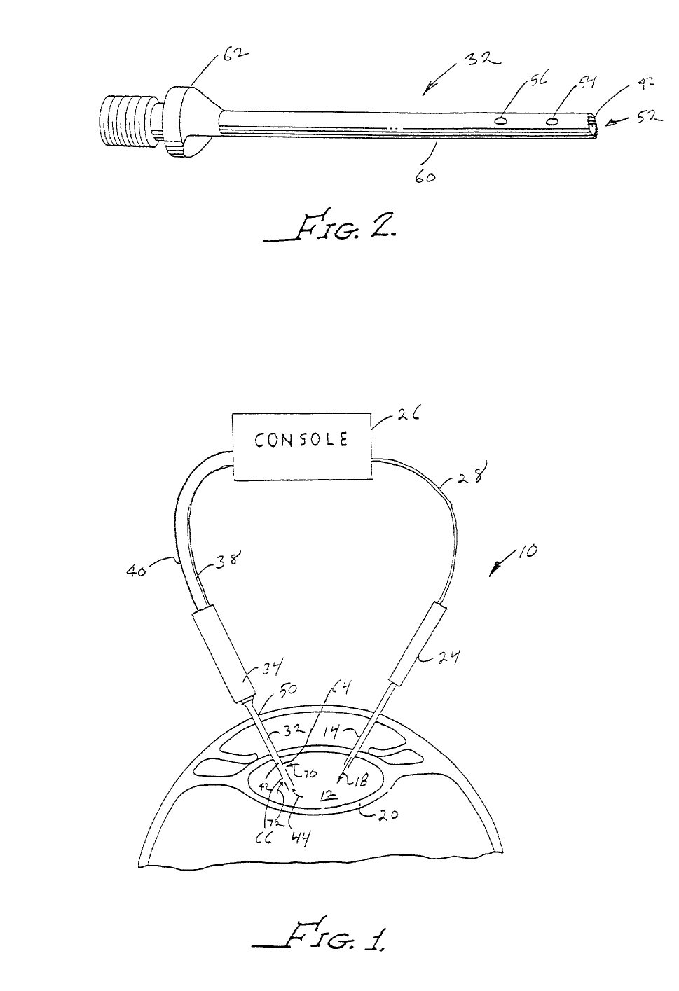

With reference to FIG. 1, there is shown apparatus 10 for the phacoemulsification of lens tissue 12 which includes a first needle 14 for introducing an irrigation fluid indicated by the arrow 18 into a lens capsule 20.

Manipulation of the needle 14 is effected through a handpiece 24 which communicates to a control console 26 through an irrigation line 28. The control console 26 may be of any suitable type as for example, one manufactured by Allergan, Inc., under the tradename Sovereign®.

Also shown in FIG. 1, as well as in FIG. 2, is a second needle 32 which is ultrasonically vibrated by a handpiece 34. Any suitable handpiece may be utilized such as for example, one sold by Allergan, Inc., under the trade name Sovereign® handpiece. The handpiece 34 is interconnected to the console 26 and control thereby through an aspiration line 38 and a power line 40 for controlling ultrasonic power delivered to the needle 32 by the handpiece 34.

The second needle 32 fragments or emulsifies the cataractic lens 12 which is then aspirated along with irrigation fluid through a lumen 42 in the needle 32 as indicated by the arrow 44.

Because the needle 32 does not include a conventional sleeve (not shown in FIGS. 1 and 2) a smaller incision or wound 50 is required. The wound size may be as small as 1.25 mm which is to be compared with conventional sleeved needles (not shown) which would require a slit or wound opening, of about 2½ to 3 mm.

As more clearly shown in FIG. 2, the needle 32 includes a primary aspiration port 52 defined by the lumen 42 and one or more secondary aspiration ports 54, 56 disposed along a length 60 of the needle 32 between the primary aspiration port 52 and a hub 62 for attachment of the needle 32 to the handpiece 34.

The secondary aspiration ports 54, 56 may be spaced apart axially from the primary aspiration port and one another as shown in FIG. 2 or, alternatively, as shown in FIG. 1 aspiration ports 64, 66 may be disposed along the needle 32 in a spaced apart radial relationship with one another resulting in aspiration of fluid from the lens capsule 20 in opposing direction as indicated by the arrows 70, 72.

The secondary aspiration ports 54, 56, 64, 66 also provide an important function in maintaining the fluid flow through the needle should the primary aspiration port 52 become excluded. This insures cooling of the needle 32 to prevent overheating thereof. Preferably, the secondary aspiration ports 54, 56, 64, 66 are disposed within 7 mm of the primary aspiration port to insure that their aspirating functionality is performed within the lens capsule 20. In order to insure that the majority of aspiration occurs through the primary aspiration port, it is preferred that the total cross-sectional area of the secondary aspiration ports 54, 56, 64,66 be no more than 10% of the cross-sectional area of the primary aspiration port 52.

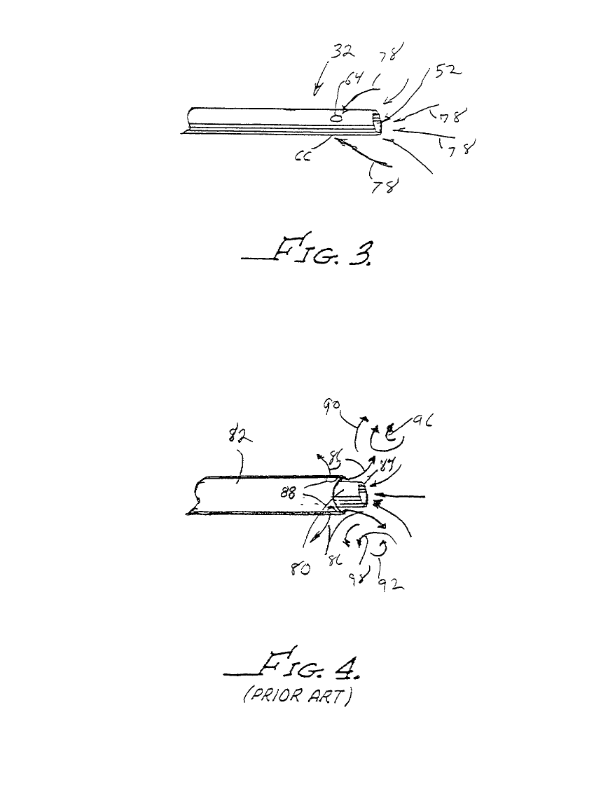

The advantages of the unsleeved needle 32 are more clearly understood with reference to FIGS. 3 and 4. FIG. 4 illustrates smooth laminar flow of aspiration fluid as shown by the arrows 78 into the primary aspiration port 52 and secondary aspiration ports 64, 66.

This is to be contrasted with a conventional phacoemulsification needle 80 which is surrounded by a sleeve 82 for the introduction of irrigation fluid proximate an aspiration port 84 as indicated by the arrows 86. Irrigation fluid is also introduced through ports 88, also indicated by arrows 86. As illustrated, aspiration of fluid as indicated by the arrows 90, 92 may be partially diverted from the port 84 before entering which causes a roiling of the fluid indicated by the arrows 96, 98. This roiling of fluid causes a “milky cloud” to appear proximate the needle 80 which interferes with the physician acuity of the needle 80 which interferes with efficient phacoemulsification of lens tissue, not shown in FIG. 4.

Although there has been hereinabove described apparatus for the phacoemulsification of lens tissue in accordance with the present invention for the purposes of illustrating in which the manner in which the invention may be used to an advantage, it will be appreciated that the invention is not limited thereto. Accordingly, any and all modifications, variations, or equivalent arrangements which may occur to those skilled in the art should be considered to be within the scope of the invention as defined in the appended claims.

What is claimed is:

1. Apparatus for the removal of lens tissue, said apparatus comprising, in combination:
a first needle for introducing an irrigation fluid into a lens capsule;
a second vibrated needle for inserting into said lens capsule and emulsifying lens tissue therein, the second needle including a lumen therethrough for aspiration of emulsified lens tissue and irrigation fluid from said lens capsule through a primary aspiration port defined by an end of said lumen; and
at least one secondary aspiration port disposed in the second needle, in a spaced apart relationship with said primary aspiration port, for aspirating fluid from the eye into said lumen.

2. The apparatus according to claim 1 wherein a cross-sectional area of the secondary aspiration port is up to 40% of a cross-sectional area of said primary aspiration port.

3. The apparatus according to claim 2 wherein the secondary aspiration port is disposed within 7 mm of said primary aspiration port.

4. The apparatus according to claim 1 further comprising multiple secondary aspiration ports in the second needle.

5. The apparatus according to claim 4 wherein a total cross-sectional area of said multiple secondary aspiration ports is up to 40% of a cross-sectional area of said primary aspirating port.

6. The apparatus according to claim 5 wherein all of said multiple secondary aspiration ports are disposed within 9 mm of said primary aspiration port.

7. The apparatus according to claim 5 wherein each of said multiple secondary aspiration ports are disposed in a spaced apart radial relationship with one another.

8. The apparatus according to claim 6 wherein each of said multiple secondary aspirations ports are disposed in a spaced apart axial relationship with one another.

9. The apparatus according to claim 7 wherein each of said multiple secondary aspiration ports are disposed in a spaced apart axial relationship with one another.

10. Apparatus for the phacoemulsification of lens tissue, said apparatus comprising, in combination:
a first needle for introducing an irrigation fluid into a lens capsule;
a second ultrasonically vibrated needle for inserting into said lens capsule and emulsifying lens tissue therein, the second needle including a lumen therethrough for aspiration of emulsified lens tissue and irrigation fluid from said lens capsule through a primary aspiration port defined by an end of said lumen; and
at least one secondary aspiration port disposed in the second needle, in a spaced apart relationship with said primary aspiration port, in order to maintain flow of emulsified lens and irrigation fluid through the second needle upon occlusion of said primary aspiration port to prevent overheating of the second needle.

11. The apparatus according to claim 10 wherein a cross-sectional area of the secondary aspiration port is up to 40% of a cross-sectional area of said primary aspiration port.

12. The apparatus according to claim 11 wherein the secondary aspiration port is disposed within 7 mm of said primary port.

13. The aspiration according to claim 10 comprising multiple secondary aspiration ports in the second needle.

14. The apparatus according to claim 13 wherein a total cross-sectional area of said multiple secondary aspiration port is up to 40% of a cross-sectional area of said primary aspiration port.

15. The apparatus according to claim 14 wherein all of the multiple secondary aspiration ports are disposed within 9 mm of said primary aspiration port.

16. The apparatus according to claim 14 wherein each of said multiple secondary aspiration ports are disposed in a spaced apart radial relationship with one another.

17. The apparatus according to claim 15 wherein each of said multiple secondary aspiration ports are disposed in a spaced apart axial relationship with one another.

18. The apparatus according to claim 16 wherein each of said multiple secondary aspiration ports are disposed in a spaced apart axial relationship with one another.
